(19) United States
(12) Patent Application Publication
(10) Pub. No.: US 2010/0234754 A1
(43) Pub. Date: Sep. 16, 2010

(54) Method and apparatus for measuring the state of hydration of hairy animals such as horses, camels, and the like

(76) Inventors: Neal S. Latman, Amarillo, TX (US); Joseph E. Batson, JR., Amarillo, TX (US); Alan Nicholson, Canyon, TX (US); Ladislado Garza, Amarillo, TX (US); Mike Floyd, Amarillo, TX (US)

Correspondence Address: Harold H. Flanders, 904 Sweetwater Cove, Round Rock, TX, 78681, US

(21) Appl. No.: 12/381,334

(22) Filed: Mar. 11, 2009

(51) Int. Cl. A61B 5/053

(52) U.S. Cl. 600/547

(57) ABSTRACT

A method and apparatus for measuring and analyzing the state of hydration of a horse, camel or other hairy animal including providing power to a transmitting circuit to transmit a signal to electrodes resulting in a potential difference across the electrodes causing a flow of current between the electrodes reflecting the impedance encountered and sending a signal reflecting such information to receiving circuits which pass that data to processing circuits which process that data with other external inputs to produce an output display reflecting an indication of the state of hydration of the animal, comprising:
means for providing a voltage difference across two or more electrodes; means for maintaining said electrodes in a fixed spatial relation and in good electrical contact with the skin of the animal without penetrating the skin of the animal on which said measurements are to be made; means for measuring the impedance of said animal in the region of or between the electrodes; means for transmitting the measured data to processing circuits which process that data with other external inputs to produce an output display reflecting an indication of the state of hydration of the animal, wherein the other external inputs include one or more of the factors for sex, age, electrode gap, and heart girth; and the output display provides indications of one or more of the outputs produced by formulations of the data reflecting the percentage of total body water, the percentage of extracellular fluid volume, the percentage of plasma and the percentage of intracellular fluid volume and the analyzing monitoring unit may be selected from the group of embodiments consisting of software in a computer, hard wired circuits, printed circuits, integrated circuits, microcircuits, microchips, silicon chips, digital chips, analog chips, hybrid chips and combinations of any or all of the members of this group.

### RIGHTS TO INVENTION UNDER FEDERAL SPONSORED RESEARCH OR DEVELOPMENT

None

### CROSS REFERENCE TO RELATED APPLICATIONS

The following applications co-owned by the same inventive entity were filed of even date herewith: CCC 0805 EQ,0806 B, and 0808 B.

### REFERENCE TO MICROFICHE APPENDIX

None

### BACKGROUND OF THE INVENTION

1. Field of the Invention

The present invention relates generally to a process, apparatus and systems for measuring the state of hydration of an animal and more particularly to a new and improved method and apparatus for use in connection with hairy animals such as horses, camels, and the like in Bioelectrical Impedance Analysis (BIA), and for such other structures, apparatus, processes, systems, and methods as may be herein disclosed.

The present invention is and may be used in connection with measurements requiring the measurement and analysis of the state of hydration as described herein and in general.

As a specific example reference is made to Bioelectrical Impedance Analysis (BIA). The initials or acronym BIA may refer to one or more of several slightly different terms all of which may be considered to be equivalent as discussed below and used herein. The meaning is almost always clear from the context of use and for the purposes of this application these terms may in general be used interchangeably. The term Bioelectrical Impedance Analysis is generally preferred and used herein as it conveys the most information in its terms. Other generally equivalent terms which may be used include Bioimpedance Analysis or Biological Impedance Analysis, Biological Impedance Interface, Electrical Bio-impedance, Electrical Impedance Analysis, and similar terms and combination of terms.

The question of the state of hydration of any patient or animal is a matter of primary and principle concern. Whenever the condition of dehydration is encountered, it must be treated before any other medical problem can be addressed. At a minimum the state of hydration must be determined before a plan for the treatment of the remaining medical problem formulated and acted upon. It is this requirement that underlies the importance of the methods and apparatus of the present invention for making the necessary determinations defining the state of hydration. It is required that these methods and their associated apparatus be quick, effective, efficient and productive with a minimum of stress to the patient as the determinations are made. In addition, the degree of hydration, within the range of normal hydration, can be significant in the performance of any animal and especially animal athletes. Therefore, the ability to attain optimal hydration is dependant on the ability to determine the degree of hydration.

A common, standard method of measuring hydration status is known as the “Indicator Dilution” method. This method measures the hydration status in terms of “Total Body Water” (TBW), “Extracellular Fluid Volume” (EFV or ECFV) and “Plasma Volume” (PV). In these procedures, indicators are injected intravenously and allowed to circulate throughout the body. When the circulation is complete, blood samples are drawn and evaluated with complicated procedures which are invasive, time consuming and expensive.

Bioelectrical Impedance Analysis (BIA) provides a way to measure hydration status non-invasively, quickly, and conveniently by measuring TBW, EFV, PV, and intra-cellular fluid volume.

BIA uses electrical potential to cause electrical currents to pass through the conductive tissue in order to measure the impedance presented. The amount of electricity conducted varies depending on the amount of water and electrolytes present in and around the tissue. These measurements take only a few minutes and produce little or no discomfort while yielding quick and accurate data relative to the status of hydration.

BIA determinations of body compositions and hydration status have been available for several years. These measures have been most commonly directed to human beings.

2. Relevant State of the Art and Description of Related Prior Art

As has been noted above, the present invention relates to the measurement and analysis of the state or degree of hydration of animals in general and especially to measurements as may be required on hairy animals such as horses, camels, and the like.

Horses are important animals having economic, agricultural, sports and entertainment significance. Various measurements may be made or desired relating to their training, working, and breeding. Similar concerns and measurements may be made on other hairy animals such as camels.

Among the measurements of particular interest are those known as Bioelectrical Impedance Analysis, although as has been noted, the present invention is not limited to such specific measurements or techniques.

Bioelectrical Impedance Analysis may be used to measure and analyze a wide range of ionic and charge transfer processes in bio-materials and biological systems in general.

As a matter of general background to the present invention, it may be helpful to note the following terms:

Electricity is the movement of electrons. Electrons have a negative charge. Free electrons will flow or move towards a positive charge or down an electrical gradient towards a less negative charge.

Amperes (Amps) is the number of free electrons flowing or moving per unit time. Sometimes this flow of electrons is referred to as “Intensity” or “I”. Sometimes this flow is referred to as “electrical current”. In many situations, it may be best to think about amperes as the amount of or volume of electrons that are moving per unit of time. One ampere=6.25×10¹⁸ electrons per second.

Conductors: The flow of electrons moves along a material or substance called a “conductor”. Some substances offer more or less resistance to the flow of electrons than others. Those that offer little resistance to the flow of electrons are considered “good conductors” A good conductor is a material that has electrons that are less tightly bound and therefore, more free to move. In a bad conductor, the electrons are more tightly bound and less free to move. A really bad conductor is called an “insulator”.

Volts represent the potential difference in charges between two points in or along a conductor. That means that there is an electrical gradient between the two points. In other words, there are more negative charges (electrons) at one point than at the other. The more positively charged point would exert an attractive force or pull on the electrons toward it. The attractive force is called an “electromotive force”.

The relationship between amperes, volts and resistance to flow of electrons may be expressed by Ohm's law: Volts=Amperes×Resistance in Ohms. All conductors offer some resistance to the flow of electrons.

A “capacitor in an electric circuit is a non-conductor (insulator, sometimes called a “dielectric”) that is sandwiched between two conductors. As the electrons flow down the conductor, it comes to the capacitor. Because the capacitor is a non-conductor, the electrons begin to pile-up on one side of it. As more negatively charged electrons accumulate, the potential electrical difference between the negative side of the capacitor and the relatively positively charged side increases. Like charges repel each other. So, as the negatively charged electrons accumulate on one side of the capacitor, the increasing negative charge on that side of the capacitor repels the negatively charged electrons on the other side of the capacitor. That results in one side of the capacitor with more electrons next to the capacitor than the other side. When the potential difference in negative electrons between the two sides is sufficiently great, the electrons on the relatively less negative side of the capacitor begin to move away from the capacitor and down the conductor. We can view a capacitor as a non-conductor that results in an increase in voltage.

Sometimes, capacitance is thought of as the amount of electrons necessary to raise the potential by a specific amount. At other times, capacitance may be thought of as the amount of electrons that can be “stored” on a surface (i.e., the negative side of the capacitor), before the electrical current moves on. Capacitance is measured in “Farads”.

A biological cell membrane is composed of a bimolecular layer of phospholipids. Lipids are poor electrical conductors. They are so poor as to be viewed as non-conductors. When an electrical current flows through the fluids in the body (a relatively good conductor) and comes to a cell membrane such as a red blood cell, the cell membrane acts as a capacitor, the capacitance of which can be measured.

Bioelectrical Impedance Analysis (BIA) measures the impedance or opposition to the flow of electrical current through body fluids. Impedance is low in lean tissue where intracellular fluid and electrolytes are primarily contained, but high in fat tissue. Impedance is generally proportional to body water volume. In practice a small constant current, typically 800 uA at a fixed frequency, for example 50 kHz, is passed between electrodes spanning the body parts in question and the voltage drop between electrodes provides a measure of impedance.

The impedance of a biological tissue is comprised of two components, the resistance and the reactance. In general, the conductive characteristics of body fluids provide the resistive component, whereas the cell membranes, acting as imperfect capacitors, contribute a frequency-dependent reactive component.

Impedance measurements made over a range of low to high (1 MHz) frequencies, allow the development of predictive equations. For example equations may relate impedance measures at low frequencies to extracellular fluid volumes and at high frequencies to total body fluid volume. This approach is known a multi-frequency bioelectrical impedance analysis (MFBIA).

The BIA measurements in general involve the measurement of:
a.) resistance in ohms {“R”}
b.) reactance in ohms {“Xc”} [basically defined as the opposition to transmission of electrical energy through a capacitor.]
c.) impedance in ohms {“Z”} [basically defined as Z=√[R²+(Xc)²] (i.e. the square root of [R squared+Xc squared]).

The above paraphrased from tutorial papers of Dr. Neal Latman and from pp. 29-32 Horowitz & Hill, The Art of Electronics (2d Ed.) Cambridge University Press, Cambridge, Mass., 1989.

The background of the present invention is more specifically provided by Forro, Mariam, Scott Cieslar, Gayle L. Ecker, Angela Walzak, Joy Hahn, and Michael I Lindenger, “Total body water and ECFV measured using bioelectrical impedance analysis and indicator dilution in horses”: J. Appl Physiol 89: 663*671,2000. which to some extent appears to teach away from the present invention, see sections on the linear regression analysis.

Also see, Fielding, C. Langdon, Gary Magdesean, Denise A. Elliott, Larry D. Cowgell, and Gary P. Carlson; “Use of multifrequency bioelectrical impedance analysis for estimation of total body water and extracellular and intracellular fluid volumes in horses”, AJVR, Vol 65, No. 3, 320,326 March 2004.

According to Coenen's review of the literature (Coenen, M. 2005. “Exercise and stress: impact on adaptive processes involving water and electrolytes.” Livestock Production Science 92:131-145.) the first use of bioelectrical impedance analysis (BIA) on horses was by Adkins (Adkins, H. A. 1996. “A preliminary study of the application of bioelectrical impedance analysis to the horse.” Warwuckshire College, Warwuck BA thesis. {cited in Coenen, 2005}) and Bartholomeeussen, L. 1996. “Estimation of body composition by deuterium dilution and bio-electrical impedance analysis in equids.” University of Aberdeen, Dept. Environmental and Occupational Medicine. MS thesis. {cited by Coenen, 2005}). Most of the research papers on the determination of horse hydration with BIA have used multiple frequency approaches. {Forro et al, J. Appl. Physiology, 2000; Fielding et al, AJVR, 2004; Waller et al, Equine Exerc. Phys., 2006 and Fielding et al, J Vet. Internal Med. 2007, McKeen, B.&M. Lindeinger, 2004. “Prediction of hydration status using multi-frequency bioelectrical impedance analysis during exercise and recovery in horses.” Equine and Comparative Exercise Physiology. 1:119-209} Based on theoretical considerations, (Lukaski, H. 1996. “Biological indexes considered in the derivation of the bioelectrical impedance analysis.” 64 (supplement):397S404S.; and also Forro et al, 2000, J Appl. Physiol. 2000) these have usually focused on two frequencies, such as 5 and 200 kHz (Forro et al, 2000) and up to as many as 50 frequencies between 5 and 1,000 kHz (Fielding et al, 2007). However, despite the theoretical advantages of using multiple frequencies, Lukaski {Lukaski, H. 1996 and Lukaski, H. 1999. “Requirements for clinical use of bioelectrical impedance analysis (BIA).” Annals of the New York Academy of Sciences. 873.72-76} has reported no apparent or significant advantage to multi-frequency BIA compared to a single frequency, such as 50 kHz in actual use.

See also U.S. Pat. No. 6,850,798 which measures animal body fat via the hooves and foot pads; U.S. Pat. Nos. 6,308,096 and 6,321,112 at their FIG. 25 and 2001/0007055 which purports to measure fatigue, see FIG. 12.

U.S. Pat. No. 6,360,124 is handheld and U.S. Pat. No. 6,400,983 which employs hand electrodes.

U.S. Pat. No. 6,477,409 measures metabolism and U.S. Pat. No. 6,487,445 utilizes calipers.

U.S. Pat. Nos. 6,490,481; 6,509,748; and 2003/0216665 employ multiple electrodes with other body data while U.S. Pat. Nos. 6,516,221 and 6,725,089 feature graphic displays.

U.S. Pat. No. 6,567,692 utilizes multiple sites, U.S. Pat. No. 6,621,013 selects body information to be evaluated.

2003/0176808 allows for multiple fat layers. 2004/0019292 permits use in identification.

2004/0171963 and 2005/0059902 focus on body composition and 2004/0236245 on muscle mass.

2005/0124909 is directed to the measurement of body fat in animals.

U.S. Pat. No. 6,978.170 focuses on electrode positioning.

2006/0094979 and 2006/0111645 utilize multiple pairs of electrode systems.

Other references of interest include:
U.S. Pat. Nos. 3,602,215; 3,851,641; 3,871,359; 3,971,365; 4,008,712; 4,116,231; 4,336,873; 4,377,170; 4,423,792; 4,144,763; 4,557,271; 4,557,271; 4,493,362; 4,578,635; 4,557,271; 4,773,492; 4,831,242; 4,831,527; 4,844,187; 4,947,862; 4,805,621; 4,895,163; 4,911,175; 4,919,145; 4,947,862; 5,063,937; 5,086,781; 5,203,344; 5,579,782; 6,088,615; 6,208,890; 5,722,396; 5,819,741; 6,004,312; 6,188,925; 6,280,396; 6,308,096; 6,354,996; 6,370,425; 6,393,317; 6,400,983; 4,949,727; 5,052,405; 5,105,825; 5,372,141; 5,458,117; 5,720,296; 5,746,214; 5,817,031; 5,840,042; 6,151,523; 6,198,964; 6,256,532; 6,265,882; 5,483,970; 5,335,667; 5,415,176; 5,435,3115; 5,449,000; 5,595,189; 5,611,351; 5,615,689; 5,749,369; 5,335,667; 5,817,031; 6,088,615; 6,292,690; 2002/0026173; U.S. Pat. Nos. 6,370,425; 6,393,317; 2002/0151815; U.S. Pat. Nos. 6,473,643'2002/0151311; U.S. Pat. No. 6,631,292; 2004/0002662; U.S. Pat. Nos. 5,088,489; 5,335,667; 5,718,850; 5,720,296; 5,729,905; 6,038,465; 6,088,615; 6,321,112; 6,398,740; 6,440,068; 6,327,495; 5,371,469; 5,483,970; 5,503,157; 5,865,763; 6,011,992; 6,339,722; 6,442,422; 6,450,955; 6,490,481; 6,487,445; 6,516,221; 6,526,315; 6,567,692; 5,579,782; 5,819,741; 6,004,312; 6,168,563; 6,280,396; 6,308,096; 6,685,654; 2004/0077968; U.S. Pat. No. 6,752,760; 2004/0260196; U.S. Pat. No. 6,865,415; 2005/0059903/; 2005/0080352; U.S. Pat. No. 6,889,076; 2005/0101875; 2005/0171451; 2005/0177060; 2005/0177062; 2005/0192488; 2005/0209528; 2006/0025701; and 2006/0094978.

While a number of Bioelectrical Impedance Analysis (BIA) systems are shown and taught by the above art they, in general, fail to recognize the importance of the proper analysis of the system as a whole as applied herein.

### BRIEF SUMMARY OF THE INVENTION

The present invention relates to the measurement and analysis of the state of hydration as applied in connection with measurements on hairy animals such as horses, camels, and the like.

The present invention may be used for information relative to racing, training, working and breeding. Whether the horse is involved in racing, rodeos, jumping or other competitions; ranch and farm work, reproductive services; or as a pet, the hydration status of the horse is important to the horse, to the owner of the horse, and to the horse's trainer and veterinarian.

A change of hydration of as little as one (1%) percent can affect the performance of the horse. Determination of the optimal hydration status for each horse is important, if the horse is to be maintained at the optimal level of hydration to ensure optimal performance. Dehydration of five (5%) percent or more is clinically significant. When dehydrated, blood pressure, heart rate, muscle function and the ability to perspire and maintain the desired body temperature is affected. Severe dehydration is life threatening.

The present invention while designed primarily for use with horses and camels can be used with other hairy animals including but not limited to cattle, hogs and dogs.

The present invention can be utilized to measure, or to be a part of a system to measure or evaluate such parameters as total body water, extra and intra cellular fluid, plasma, lean muscle mass, fat, the extent of marbling, phase angles, and general, overall health. The present invention can provide both preventative analysis and the detection of contamination and out of balance states of hydration.

### Objects

Pursuant to the foregoing, it may be regarded as an object of the present invention to overcome the deficiencies of and provide for improvements in the state of the prior art as described above and as may be inherent in the same or as may be known to those skilled in the art.

It is a further object of the present invention to provide a process and any necessary apparatus for carrying out the same and of the forgoing character and in accordance with the above objects which may be readily carried out with and within the process and with comparatively simple equipment and with relatively simple engineering requirements.

Still further objects may be recognized and become apparent upon consideration of the following specification, taken as a whole, in conjunction with the appended drawings and claims, wherein by way of illustration and example, an embodiment of the present invention is disclosed related to hairy animals such as horses and camels, and the bioelectrical impedance analysis of the same.

As used herein, any reference to an object of the present invention should be understood to refer to solutions and advantages of the present invention which flow from its conception and reduction to practice and not to any a priori or prior art conception.

The above and other objects of the present invention are realized and the limitations of the prior art are overcome by providing a new and improved apparatus, methods and processes applicable to measurements to be made on hairy animals such as horses and camels.

### Technical Problems to be Solved

The need for a system to provide accurate, reliable and repeatable measurements has long existed and been an unfulfilled need prior to the invention of the present apparatus and process.

In particular, the uneven topographic surfaces presented by certain animals such as horses and the like which may have abundant hair coats over uneven muscles and bone structures have long presented a problem of obtaining accurate and reproducible measurements in various electrical systems including those directed to bioelectrical impedance analysis.

The body proportions, relative amounts of different tissues, and tissue characteristics of the animals this invention addresses are significantly different from humans. Life style information that can be incorporated into human BIA determination is not available for these animals. Because of the various locations and situations in which the hydration status or degree of hydration must be determined in these animals, some of the variables, such as body weight, used in human BIA determinations cannot be used with these animals. Therefore, the characteristics of BIA measurements are different for these animals than humans.

### BRIEF DESCRIPTION OF THE DRAWINGS AND THEIR SEVERAL VIEWS

The above mentioned and other objects and advantages of the present invention and a better understanding of the principles and details of the present invention will be evident from description taken in conjunction with the appended drawings.

The drawings constitute a part of this specification and include exemplary embodiments of the present invention, which may be embodied in various forms. It is to be understood that in some instances various aspects of the invention may be shown as exaggerated, reduced, or enlarged or otherwise distorted to facilitate an understanding of the present invention.

In the drawings appended hereto:

FIG. 1 is a schematic flow sheet showing the system of the present invention.

In the accompanying drawings, like elements are given the same or analogous references when convenient or helpful for clarity. The same or analogous reference to these elements will be made in the body of the specification, but other names and terminology may also be employed to further explain the present invention.

### GENERAL DESCRIPTION OF THE INVENTION, DETAILED DESCRIPTION OF PREFERRED EMBODIMENTS OF AND BEST MODE FOR CARRYING OUT THE PRESENT INVENTION

As described in the background set forth above, BIA are of course known. However, such measurements on a horse, camel or the like present a number of additional problems. The density and length of the hair can be problematical. The uneven topography of horses requires special considerations and electrode configurations. The body proportions, relative amounts, and characteristics of the animals this invention addresses are significantly different from humans. Therefore, the characteristics of BIA measurements are different for these animals than humans.

It is common for human and other BIA determinations to require the use of the body weight. Scales are not always available to measure the weight of horse and camels. “Life Style” considerations are more difficult to evaluate in the case of horses.

As the present invention relates to methods and apparatus for use on animals in connection with Bioelectrical Impedance Analysis (BIA) and other electrical measurements. The electrical measurements may be directed to any of a number of areas of electrical activities either of endogenous or exogenous origin. The preferred electrodes are for use on hairy animals such as a horses, camels, and/or the like. The electrodes are of the surface electrode type which do not, and are not intended to, penetrate the skin. The electrodes systems are intended to overcome problems in the prior art as noted above. They will provide a defined distance between the surface contact points of the electrodes. They will also be flexible enough to adjust to uneven surfaces, contours and topology of the animal. The electrodes and their holders are designed to provide for movement of the electrodes to overcome surface topography and to provide the other needs in providing accurate and reproducible measurements.

The present invention may be used and/or adapted for use in or in connection with any Bioelectrical Impedance System. One preferred system is that known as a tetra-polar system which usually will employ two (2) electrodes at each end of a hairy animal.

The connecting rod between electrodes is an insulating flexible rod on the order of 5-10 cm in length. The rod may be a relatively large diameter wire or rod of copper, carbon, silver, gold, platinum or other suitable flexible material. The rod may be a conductor suitably insulated or a non-conductive, electrically insulating material.

The electrodes may be coated with a conducting cream or gel to improve contact with the animal skin. The electrode holder may optionally include a compartment for containing and dispensing such cream or gel.

The electrodes may have attachment clips of various known designs.

The electrodes may be covered with elastomeric conformable or malleable conductive material having physical properties similar to some known silicone products. Some of the conformable materials may be assisted in their properties by the heat of the animals' skin.

The system is not limited to hard wired transmission of its power voltage or signals.

The flexible rods may consist of insulating plastic tubing surrounding a concentric internal carbon fiber or other suitably flexible rod.

For further details of the electrodes and the electrode holders for such electrodes, see the co-pending patent application designated CCC 0805 EQ, entitled ELECTRODE HOLDER FOR USE ON HAIRY ANIMALS SUCH AS HORSES, CAMELS, AND THE LIKE, filed contemporaneously herewith, which is hereby incorporated by reference with the intention that it be treated and understood as fully as if set forth herein in its entirety.

In general, electrodes are positioned and attached by a flexible rod which is an insulating rod or a coated rod of insulating materials around a conductive or non-conductive rod or core of carbon or other suitable material.

The electrodes have detents or hooks to allow an elastomeric material (such as an elastic bandage) to be attached to electrodes and wrapped around a limb or other body part of the animal. The rod is attached to the electrode by clips which engage the rod and are held in place by the insulating material on the rod. The clip is in electrical contact with the electrode.

The electrode is curved to conform to the animal leg or other body part.

Other electrical clips and attachments may be clipped to a clip or strap which is attached to the electrode. The electrode may be hardwired to the appropriate electrical input for BIA or other measurements. Additionally the electrode holder can provide a base on which can be mounted an electromagnetic energy power supply (such as electricity) and/or an electromagnetic signal (such as radio or light) receiver and/or transmitter which allows the system to work wirelessly. A wireless arrangement makes the entire BIA process a lot easier and simpler by eliminating the need for using wires between the instrument and the animal and thereby, also, reducing the dangers and problems associated with using wires around the animal.

For a further understanding of the nature, function, and objects of the present invention, reference should now be made to the following detailed description taken in conjunction with the accompanying drawings. Detailed descriptions of the preferred embodiments are provided herein, as well as, the best mode of carrying out and employing the present invention. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure, method, process, or manner. The practice of the present invention is illustrated by the following examples, which are deemed illustrative of both the process taught by the present invention and of the results yielded in accordance with the present invention.

Functionally and in operation the present invention may be seen as a tetra-polar Bioelectrical Impedance Analysis System in which two surface electrodes at each end are employed to facilitate the impedance measurement. This system is intended for use primarily on hairy animals such as horses, camels and the like.

The measurement of impedance is known and any of the many suitable systems may be used in the context of the present invention.

The electrodes need to be a set distance apart in order to provide reproducible measurements. In order to provide a good fit, vertical and horizontal independent movement is desired and provided.

Clips may be used to make good electrical contact with the electrode plates. Wireless transmission systems may be employed in connection with the present invention.

A variety of connecting rods, usually on the order of 5 to 10 cm, may be employed. Each rod needs to be suitably insulated and rods of various lengths provide for desired adjustment in length within the system.

Good electrical contact may be further facilitated by the use of known conducting creams and gels. Such creams and gels may be provided by use of a holder within or attached to the electrode structure.

Added flexibility may be obtained, if desired by use of ball and socket joints.

To assist in the understanding of the present invention the following example should be considered.

### EXAMPLE

Five (5) healthy adult horses were used in a randomized crossover design for this example. The horses were kept in stalls and fed their usual rations of grass, hay, and grain. Each horse had its body fluids measured while normally hydrated and again after the administration of furosemide, following which they were not allowed access to water, salts, or feed until all measurements were complete. Hydration indicators for the indicator dilution studies were injected two (2) hours after the furosemide treatment.

The hydration status of the total sample size of ten (10) horses was measured using standard reference indicator dilution methods.

Total Body Water (TBW) was measured with the administration of deuterated water (110 mg/kg body infused as a 50% v/v solution of 0.9% NaCl).

Extracellular Fluid Volume (EFV) was measured with the administration of sodium thiocyanate (22 mg/kg body weight infused as a 20% w/v) solution. Venous blood samples were taken at time 0 (immediately prior to infusion) and at 10,20,30,45,60,90, and 120 minutes post-infusion.

Plasma volume was measured with administration of indocyanine green dye (0.25 mg/kg body weight). Venous blood samples were obtained every twenty (20) seconds for five (5) minutes.

Intracellular Fluid Volume (% ICFV) was calculated by subtracting the percentage of Extracellular Fluid Volume (% ECFV) from the percentage Total Body Water (% TBW) [each as obtained from the procedures as set forth above), (i.e., % TBW−% ECFV=% ICFV)

Simultaneous measurements with a BIA were performed. The variables measured on each horse included:

1. Age

2. Sex Status

3. Breed

4. Weight

5. Forearm Length

6. Gaskin Length

7. Elbow to Stifle Length

8. Heart Girth

9. Electrode Gap

10. Torso Volume (inches³)

11. BIA Resistance [R}

12. BIA Reactance [Xc]

13. BIA Impedance [Z]

14. BIA Phase Angle

15. BIA Torso Volume [Electric Volume]

The BIA measurements were determined with a “Quantum X” model BIA instrument manufactured by RJL Systems, Inc. Clinton Township, Mich., USA.

A tetrapolar arrangement of electrodes was made by placing two (2) pairs of surface electrodes on the skin. Each pair consisted of a “Signal Electrode” and a “Detecting Electrode” The signal and detecting electrodes were spaced ten (10) cm apart with the signal electrodes distal to the detecting electrodes. One pair of electrodes was placed over the ulnaris lateralis muscle on the forearm. The second pair of electrodes was placed over the gastrocnemius muscle on the gaskin. Each pair of small, rectangular, silver-plated electrodes was attached to an electrode holder that assured the proper ten (10) cm spacing and permitted sufficient movement to compensate for the uneven topography of the horse anatomy. A sufficient quantity of electrical conducting gel was placed between the electrodes and the horse skin to ensure a good electrical contact.

The BIA signal of 425 microamps at 50.0 kH (=/−1%) was sent from the signaling electrodes. The measurements from the detecting electrodes were transmitted to the Quantum X BIA instrument and recorded.

As indicated above, the hydration status of each horse was measured under normal hydrated and under furosemide treated, relative dehydration conditions. The calibration of the Quantum X BIA instrument was checked before and after each measurement to ensure instrumental accuracy.

Data analysis was performed with the Statgraphic Plus 5 statistical and graphing program for computers. The statistical analysis included sub-programs for descriptive, correlation coefficient, regression, regression model selection, and multiple regression analysis.

The equations or algorithms to provide a P value<0.05 indicating that a statistically significant relationship exists between the variables at the 95% confidence level has been sought and obtained from the fifteen (15) variables listed above.

The % TBW is obtained by a constant; a value for Sex Status, where 1=Male and 2=Female; the horse's age in years; the reactance in ohms; the impedance in ohms, and the electric volume, where the electric volume is the electrode gap [EG] squared divided by the resistance [R].

EV=[EG]²/R

The electrode gap is in inches and the resistance in ohms.

The electrode gap is the Forearm Length [FL] plus the Gaskin Length [GL] plus the Elbow to Stifle Length [ESL] where all electrode gap measurements are in inches.

EG=FL+GL+ESL

The “forearm” is the generally well muscled segment of the horse's front leg extending from the elbow to the knee.

The “Gaskin Length” is the region between the Stifle and the Hock of the horse's back legs.

The “Elbow to Stifle Length” is the substantially horizontal distance from the Elbow to the Stifle where the Elbow is the bony prominence lying against the chest at the beginning of the forearm and the Stifle is the joint at the end of the thigh functionally corresponding to the human knee.

As has been previously discussed above, bioelectrical impedance analysis (BIA) is based on the measurement of the impedance or opposition to the flow of electrons or an electrical current through the body fluids which are contained primarily in the lean and fat tissue of the body. In general, impedance is low in the lean tissue, where intracellular fluids and electrolytes are primarily contained, but high in fat tissue. Impedance is thus, in general, proportional to body water volume (TBW or total body water).

In making a bioelectrical impedance analysis, a small constant current, typically 800 μA, at a fixed frequency, such as 50 kHz, is passed between electrodes spanning the body and the voltage drop between the electrodes is taken as a measure of the impedance.

The impedance of a biological tissue may be viewed as being comprised of two (2) components: the resistance and the reactance. The conductive characteristics of the body fluids provide the resistive component and the cell membranes, acting as imperfect capacitors, contribute a frequency-dependent reactive component.

Impedance measurements may be made at a fixed frequency or over a range of low to high frequencies (on the order of 1 MHz) allowing the development of predictive equations relating the impedance measures at low frequencies to point to extracellular fluid volume and at high frequencies to total body fluid volume. Such an analysis is known as multi-frequency bioelectrical impedance analysis (MFBIA).

The electrodes employed as well as their holders may be important factors affecting accuracy and reproducibility. See for example the co-pending application if the present inventors filed concurrently herewith directed to the preferred electrode holders.

A multiple linear regression analysis was performed to provide the following relationships between % TBW (percentage body water in liters/kg.) and five (5) independent variables each of which at the 95% confidence level.

$$\% TBW = Constant {C}_{1} + {P}_{1} Sex Factor + {P}_{2} Age + {P}_{3} Reactance + {P}_{4} Impedance + {P}_{5} Electric Volume .$$

A particularly preferred embodiment is:

$$\% TBW = 7.64 - 12.80 Sex Factor - 0.42 Age - 1.98 Reactance + 1.08 Impedance + 0.71 Electric Volume .$$

A more general preferred formulation is:

$$\% TWB = [ 66.15 to - 50.87 ] - [ 8.96 to 16.64 ] Sex Factor - \\ [ 0.13 to 0.71 ] Age + [ 0.28 to - 4.24 ] Reactance + \\ [ 1.65 to 0.52 ] Impedance + [ 1.16 to 0.26 ] Electric Volume .$$

A multiple linear regression analysis was performed to provide the following relationships between % Extracellular fluid (% ECFV) [% body weight in liters/kg] and five (5) independent variables each of which at the 95% confidence level.

$$\% ECFV = Constant {C}_{2} + {P}_{6} Sex Factor + {P}_{7} Age + {P}_{8} Resistance + {P}_{9} Reactance + {P}_{10} Electric Volume .$$

A particularly preferred embodiment is:

$$\% ECFV = 90.91 - 5.68 Sex Factor - 0.76 Age + 0.53 Resistance - 4.00 Reactance - 0.24 Electric Volume .$$

A more general preferred formulation is:

$$\% ECFV = [ 212.02 to - 30.21 ] + [ 1.68 to - 13.04 ] Sex Factor - [ 0.08 to 1.44 ] Age + [ 1.46 to - 0.39 ] Resistance - [ 0.76 to 7.23 ] Reactance + [ 0.67 to - 1.14 ] Electric Volume .$$

A multiple linear regression analysis was performed to provide the following relationships between % Plasma [% body weight in liters/kg] and five (5) independent variables each of which at the 95% confidence level, where the plasma consists of water in which numerous chemical compounds, solids, liquids, and gases are dissolved. Included are water electrolytes, sugar, glucose, proteins, non-protein nitrogenous compounds, fats, bile pigment (bilirubin) and gases.

$$\% Plasma = Constant {C}_{3} + {P}_{11} Sex Factor + {P}_{12} Age + {P}_{13} Resistance + {P}_{14} Reactance + {P}_{15} Impedance .$$

A particularly preferred embodiment is:

$$\% Plasma = 4.73 - 0.85 Sex Factor - 0.05 Age - 0.09 Resisance - 0.21 Reactance + 0.13 Impedance .$$

A more general preferred formulation is:

$$\% Plasma = [ 7.97 to 1.48 ] - [ 0.12 to 1.57 ] Sex Factor + [ 0.03 to - 0.13 ] Age + [ 0.21 to - 0.39 ] Resistance + [ 0.30 to - 0.73 ] Reactance + [ 0.39 to - 0.12 ] Impedance .$$

A multiple linear regression analysis was performed to provide the following relationships between % Intracellular fluid volume (% ICFV) [% body weight in liters/kg] and seven (7) independent variables each of which at the 95% confidence level.

$$\% ICFV = Constant {C}_{4} + {P}_{16} Sex Factor + {P}_{17} Age + {P}_{18} Reactance + {P}_{19} Hearth Girth + {P}_{20} Impedance + {P}_{21} \cdot Phase Angle + {P}_{22} Electric Volume .$$

A particularly preferred embodiment is:

$$\% ICFV = 138.41 - 17.32 Sex Factor + 1.61 Age - 14.02 Reactance - 5.03 Hearth Girth + 4.69 Impedance + 8.30 Phase Angle + 1.46 Electric Volume .$$

A more general preferred formulation is:

$$\% ICFV = [ 448.71 to - 171.89 ] - [ 5.48 to 29.16 ] Sex Factor + [ 3.60 to - 0.38 ] Age + [ 1.13 to - 29.18 ] Reactance - [ 0.07 to 9.99 ] Hearth Girth + [ 8.09 to 1.28 ] Impedance + [ 20.87 to - 4.27 ] Phase Angle + [ 2.83 to 0.10 ] Electric Volume .$$

In connection with the above formulation, the heart girth is the measurement around the horse at the withers and behind the front legs which is, in general proportionally related to the horse's weight.
The phase angle is the difference between the phase of a sinusoidally varying quantity and the phase of a second quantity which varies sinusoidally at the same frequency. In BIA the difference in phase between the components affected by the capacitive elements (in the reactive component) and those without a capacitive effect (in the resistive component).

An alternative formulation for % ICFV is:

% ICFV=% TBW−% ECFV

A preferred formulation is:

$$\begin{matrix}\% ICFV = \sum [ 7.64 - 12.80 Sex Factor - 0.42 Age - \\ 1.98 Reactance + 1.08 Impedance + \\ 0.71 Electric Volume ] - \\ \sum [ 90.91 - 5.68 Sex Factor - 0.76 Age + \\ 0.53 Resistance - 4.00 Reactance - \\ 0.24 Electrical Volume ] . \\ = - 83.27 - 7.12 Sex Factor + .034 Age + \\ 2.02 Reactance - 0.53 Resistance + \\ 1.08 Impedance + 0.95 Electric Volume .\end{matrix}$$

A more general preferred formulation is:

$$\% ICFV = \sum \% TBW - \sum \% ECFV \sum [ 66.15 to - 50.87 ] - [ 8.96 to 16.64 ] Sex Factor - [ 0.13 to 0.71 ] Age + [ 0.28 to - 4.24 ] Reactance + [ 1.65 to 0.52 ] Impedance + [ 1.16 to 0.26 ] Electric Volume - \sum [ 212.02 to - 30.21 ] + [ 1.68 to - 13.04 ] Sex Factor - [ 0.08 to 1.44 ] Age + [ 1.46 to - 0.39 ] Resistance - [ 0.76 to 7.23 ] Reactance + [ 0.67 to - 1.14 ] Electric Volume .$$

From the above, it can be seen that BIA was able to accurately determine the hydration states of a horse. The results closely and consistently correlated with the standards for each particular aspect of the horse's hydration status The measurements were done quickly and minimally invasively.

The methods and the associated apparatus described herein can be used to quickly, accurately, efficiently and minimally invasively determine the hydration status of horses, camels and other hairy animals.

In the above paragraphs, the term “minimally invasively” has been used to refer to the methods and apparatus of the present invention. These methods and apparatus are frequently and commonly referred to as “non-invasive”. However, since the present invention puts electrical current into the body it is to that extent “invasive” and thus, for the purpose of this description, it is termed “minimally invasive”.

Based on the standard reference indicator dilution method the sample provided a relatively wide range of hydration values. The % total body water (TBW) had a mean of 68.10% and a range of 62.35 to 73.61%. The % extra-cellular fluid volume (ECFV) had a mean of 29.45% and a range of 16.29 to 34.91%. The % plasma (PV) had a mean of 2.54% and a range of from 1.89 to 3.19%. The % intra-cellular fluid volume (ICFV) had a mean of 37.60% and a range of 33.85 to 41.43%.

The horse sample ranged from 5 to 16 years of age, and included 40% mares and 60% geldings. Breeds included 80% Quarter Horses and 20% Thoroughbreds.

The mean EFV as measured with the sodium thiocyanate dilution technique was 312.2 ml/kg or 30.72% of the body weight in untreated horses and 268.9 ml/kg or 26.01% of the body weight of horses treated with furosemide. This correlates to a 15.34% drop in EFV due to furosemide treatment. The mean PV as measured with the indocyanine green dilation technique was 26.54 ml/kg or 2.52% of the body weight in untreated horses and 24.11 ml/kg or 2.56% of the body weight of horses treated with furosemide. This correlates to a 1.84% increase in PV with furosemide treatment. The mean TBW as measured with the deuterated water dilution technique was 684.49 ml/kg or 68.55% of the body weight in untreated horses and 643.96 ml/kg or 64.47% of the body weight of horses treated with furosemide. This correlates to a 5.95% decrease in TBW due to furosemide treatment.

The multiple regression model selection program was used to select the smallest combination of variables that provided the largest significant adjusted coefficient of determination (adjusted r²) with the smallest Mallow's Cₚ statistic.

A comparison by multiple regression analysis of the reference indicator dilution standard method determinations of hydration status to those derived from BIA and related variables is set forth below.

The % total body water (TBW) has a correlation coefficient (r) of 0.989, a standard error of the estimate (S.E.E.) of 0.92%, a clinical accuracy range (accuracy for 95% of measurement) of ±1.8%, and a mean absolute error (MAE) of 0.54%.

The % extra-cellular fluid volume (ECFV) has a correlation coefficient (r) of 0.91, a standard error of the estimate (S.E.E.) of 2.54%, a clinical accuracy range (accuracy for 95% of measurement) of ±5%, and a mean absolute error (MAE) of 1.69%.

The % plasma volume (PV) has a correlation coefficient (r) of 0.90, a standard error of the estimate (S.E.E.) of 0.27%, a clinical accuracy range (accuracy for 95% of measurement) of ±0.54%, and a mean absolute error (MAE) of 0.14%.

The % intra-cellular fluid volume (ICFV) has a correlation coefficient (r) of 0.97, a standard error of the estimate (S.E.E.) of 1.18%, a clinical accuracy range (accuracy for 95% of measurement) of ±2.4%, and a mean absolute error (MAE) of 0.42%.

The examples set forth above used a single frequency of 50 kHz. Of course, multiple frequencies can be used as a part of the present invention.

In accordance with the teachings of the present invention the signal received from the electrode(s) can be processed in conjunction with data entered into the unit according to one or more of the above formulations of factors to produce the desired output information.

The analyzing monitor unit will transmit a signal to one or more electrodes which will cause a flow of electrons to pass through the tissues of an animal so that the impedance of those tissues can be measured and transmitted to the electronic circuits of the analyzing monitoring unit where that information together with the entered data can be used to produce the desired measured quantities.

The electrical/electronic circuits may be hardwired circuits containing known digital or analog elements or both. The analyzing monitoring unit of the present invention can also employ appropriate software to the same ends in a general purpose digital computer, a special purpose analog computer or in a hybrid unit containing digital and analog elements.

The analyzing monitoring unit of the present invention may take the form of a printed circuit or integrated circuit, microcircuit, microchip, silicon chip or an item known simply as a chip. Any and all of the above may be analog, digital, or mixed, hybrid signal devices, where a hybrid or mixed signal device has both analog and digital elements on the same chip and/or within the same circuits.

Illustrative of such circuit devices are U.S. Pat. Nos. 3,029,366; 3,138,743; 3,138,747; 3,261,081, and 3,434,015. The electronic elements and circuits for carrying out the present invention are all well known to those skilled in the arts.

The electrode(s) and electrode holder for use within the present invention are disclosed in a concurrently filed application by the present inventors and is commonly co-owned.

The electronic components and circuits as set forth herein can, within the scope of the present invention be replaced in whole or part by alternative means including but not limited to mechanical, pneumatic means, optical chips, molecular, holographic and quantum elements and/or computers.

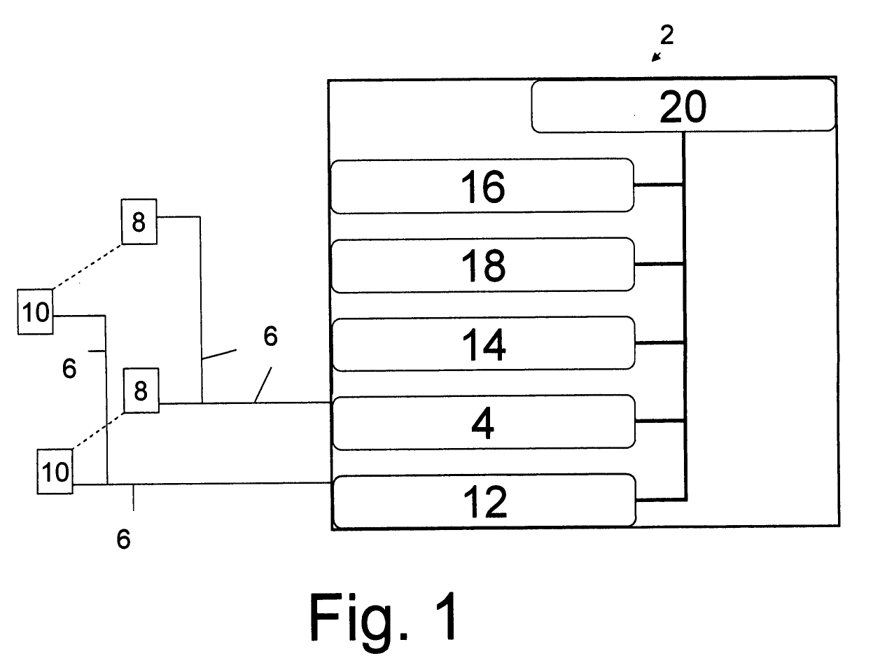

In operation, the monitor 2 of the present invention, as is shown in FIG. 1, powered by power source 20 transmits a signal as described herein from it by transmitting circuits 4 via wires 6 (or by wireless means) to an electrode 8 (or electrodes) mounted within an electrode holder (not shown in detail but disclosed in a co-pending application of the present inventors which is co-owned and filed concurrently herewith). The signal creates a potential difference between the electrodes which creates a current flow between the electrodes reflecting the impedance encountered . A signal reflecting that impedance is transmitted from electrode 10 to the receiving circuits 12. The resulting signal is passed to the memory, logic, processing and adding circuits of the processing unit 14. External inputs (such as the sex, age, electrode gap and the like) may be submitted by input means 16. This input data has been or is entered into the processing unit 14 where it is processed so that the desired information may be displayed by and at the output display 18.

### Alternatives and Alternative Embodiments

While throughout this description, we have referred to various materials, chemicals, and apparatus as being presently preferred, it will be clear to one skilled in the art that other materials, chemicals, apparatus, methods, processes, steps and embodiments may be employed which will also provide the advantages as herein set forth in connection with the present invention. The present invention is not limited to the representative examples disclosed herein. Moreover, the scope of the present invention covers conventionally known variations and modifications to the system and the components described herein, as would be known by those skilled in the art. Such variations and equivalents are intended to be within the scope of the present invention. Accordingly, the invention is to be broadly construed and is to be limited only by the scope and spirit of the claims appended hereto.

To provide a description of the present invention that is both concise and clear, various examples of ranges have been set forth herein and in all cases should be read as though expressly identified with the phrase “including all intermediate ranges and combinations thereof”. Examples of specific values (e.g., ohms, ° C., μm, kg/L, volts, amps, current, intensity, etc.) that can be within a cited range by the reference to “including all intermediate ranges and combinations thereof” include 0.000001, 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1050, 1100, 1150, 1200, 1250, 1300, 1350, 1400, 1450, 1500, 1600, 1700, 1800, 1900, 2000, 3000, 4000, 5000, 6000, 7000, 8000, 9000, 10,000, or more and so forth.

The above conventions may be understood by means of a number line a common element of elementary mathematics. It can be constructed by marking off two points: zero (the origin) and one (1). The distance from 0 to the point 1 is called the unit segment. The distance between all consecutive whole numbers is the same. When measurements fall somewhere between whole numbers, we may describe the situation in terms of a fractional length or in decimal terms of tenths, hundredths, thousandths and so forth. For example, if a measurement falls between 4 and 5, we may find that it is closer to 4.3 than 4.4. If we want more precision (and it is appropriate), we may continue to “zoom in” in which case we move more decimal places to the right. For numbers less than 5 in the relevant place, one may round down and for numbers greater than 5 one may round up. When the relevant place contains a 5, the rule is to round so that the last nonzero digit is an even number. Whenever a range is given herein the above rules are intended to apply and the range is intended to cover all points on the number line from the lowest number to be rounded to the bottom of the range to the highest number to be rounded to the top of the range.

General ranges and the usual definitions for significant figures for each type of unit (e.g., ohms, %, ° C., μm, kg/L), are contemplated. Examples of values that can be within a cited percentage range, as applicable, include 0.001% to 100%, including all intermediate ranges and combinations thereof. Examples of values that can be within a thickness range (e.g., coating and/or film thickness upon a surface), as applicable, in micrometers (“μm”), that can be within a cited range include 1 μm to 2000 μm, including all intermediate ranges and combinations thereof. Similar examples may be understood to apply to all of the units and systems of units mentioned above, such as ohms and the like or otherwise discussed below.

The following comments are intended to apply to all units and their conversions to whatever system of units including but not limited to length (m), mass (kg), time (s), speed, force, work, energy, heat, pressure including, but not limited to, angular frequency or velocity (radian/second), reactance (ohm), resistance (ohm) capacitance (farads), charge (coulomb), current (ampere), electromotive force (volt), work or energy joule), force (Newton), frequency (Hertz), inductance (Henry), magnetic field (B, Tesla), Magnetic flux (Weber), potential (volt) power (watt), etc.

Specific units from one or more of the following systems may be used including but not limited to S.I., m.k.s. practical units; Gaussian units; Heaviside-Lorentz units; electrostatic units, and/or electromagnetic units.

In addition to the standard units the micron (μ=10⁻⁶ m) and Angstrom (Å=10⁻¹⁰ m) are frequently used and may be used herein.

The electrode may be hardwired to the appropriate electrical input for BIA or other measurements. Additionally the electrode holder can provide a base on which can be mounted an electro-magnetic energy power supply (such as electricity) and/or an electro-magnetic signal (such as radio or light) receiver and/or transmitter which allows the system to work wirelessly. A wireless arrangement makes the entire BIA process a lot easier and simpler by eliminating the need for using wires between the instrument and the animal and thereby, also, reducing the dangers and problems associated with using wires around the animal.

### Summary

The present invention makes it possible to estimate the % total body water in horses to within 1.8% in 95% of its predictions with a mean absolute error of only 0.5%.

The present invention includes a method and apparatus for measuring and analyzing the state of hydration of a horse, camel or other hairy animal including providing power to a transmitting circuit to transmit a signal to electrodes resulting in a potential difference across the electrodes causing a flow of current between the electrodes reflecting the impedance encountered and sending a signal reflecting such information to receiving circuits which pass that data to processing circuits which process that data with other external inputs to produce an output display reflecting an indication of the state of hydration of the animal, comprising:
means for providing a voltage difference across two or more electrodes; means for maintaining said electrodes in a fixed spatial relation and in good electrical contact with the skin of the animal without penetrating the skin of the animal on which said measurements are to be made; means for measuring the impedance of said animal in the region of or between the electrodes; means for transmitting the measured data to processing circuits which process that data with other external inputs to produce an output display reflecting an indication of the state of hydration of the animal, wherein the other external inputs include one or more of the factors for sex, age, electrode gap, and heart girth; and the output display provides indications of one or more of the outputs produced by formulations of the data reflecting the percentage of total body water, the percentage of extracellular fluid volume, the percentage of plasma and the percentage of intracellular fluid volume and the analyzing monitoring unit may be selected from the group of embodiments consisting of software in a computer, hard wired circuits, printed circuits, integrated circuits, microcircuits, microchips, silicon chips, digital chips, analog chips, hybrid chips and combinations of any or all of the members of this group.

The present invention may employ an electrode holder comprising a pair of electrodes connected by a flexible, insulating rod which controls the position of the electrodes with respect to each other.

It is noted that the embodiment described herein in detail for exemplary purposes is, of course, subject to many different variations in structure, design, application, and methodology. Because many varying and different embodiments may be made within the scope of the inventive concepts herein taught, and because many modifications may be made in the embodiment herein detailed in accordance with the descriptive requirement of the law, it is to be understood that the details herein are to be interpreted as illustrative and not in a limiting sense. It will be understood in view of the instant disclosure, that numerous variations of the invention are now enabled to those skilled in the art. Many of the variations reside within the scope of the present teachings. It is not intended to limit the scope of the invention to the particular forms set forth, but on the contrary, it is intended to cover such alternatives, modifications, and equivalents as may be included within the spirit and scope of the teachings and claims of the present invention. Accordingly, the invention is to be broadly construed and is to be limited only by the spirit and scope of the claims appended hereto.

What is claimed is:

1. A method for determining and monitoring the state of hydration of a horse, camel or other hairy animal by providing power to a transmitting circuit to transmit a signal to electrodes resulting in a potential difference across the electrodes causing a flow of current between the electrodes reflecting the impedance encountered and sending a signal reflecting such information to receiving circuits which pass that data to processing circuits which process that data with other external inputs to produce an output display reflecting an indication of the state of hydration of the animal, comprising
(a) providing a voltage difference across two or more electrodes;
(b) maintaining said electrodes in a fixed spatial relation and in good electrical contact with the skin of the animal without penetrating the skin of the animal on which said measurements are to be made;
(c) measuring the impedance of said animal in the region of or between the electrodes;
(d) transmitting the measured data to processing circuits which process that data with other external inputs to produce an output display reflecting an indication of the state of hydration of the animal, wherein the other external inputs include one or more of the factors for sex, age, electrode gap, and heart girth; and the output display provides indications of one or more of the outputs produced by formulations of the data reflecting the percentage of total body water, the percentage of extracellular fluid volume, the percentage of plasma and the percentage of intracellular fluid volume and the analyzing monitoring unit may be selected from the group of embodiments consisting of software in a computer, hard wired circuits, printed circuits, integrated circuits, microcircuits, microchips, silicon chips, digital chips, analog chips, hybrid chips and combinations of any or all of the members of this group.

2. The method of claim 1 wherein the circuits employed are at least in part hard wired.

3. The method of claim 1 wherein the circuits employed are at least in part printed or integrated circuits.

4. The method of claim 1 wherein the circuits employed are at least in part contained on a chip.

5. The method of claim 1 wherein the percentage of total body water is determined by the formulation:
[66.15 to −50.87]−[8.96 to 16.64] Sex Factor−[0.13 to 0.71] Age+[0.28 to −4.24] Reactance+[1.65 to 0.52] Impedance+[1.16 to 0.26] Electric Volume.

6. The method of claim 5 wherein the percentage of total body water is determined by the formulation:
7.64−12.80 Sex Factor−0.42 Age−1.98 Reactance+1.08 Impedance+0.71 Electric Volume.

7. The method of claim 1 wherein the percentage of extracellular fluid is determined by the formulation:
[212.02 to −30.21]+[1.68 to −13.04] Sex Factor−[0.08 to 1.44] Age+[1.46 to −0.39] Resistance−[0.76 to 7.23] Reactance+[0.67 to −1.14] Electric Volume.

8. The method of claim 7 wherein the percentage of extracellular fluid is determined by the formulation:
90.91−5.68 Sex Factor−0.76 Age+0.53 Resistance−4.00 Reactance−0.24 Electric Volume.

9. The method of claim 1 wherein the percentage of plasma is determined by the formulation:
[7.97 to 1.48]−[0.12 to 1.57] Sex Factor+[0.03 to −0.13] Age+[0.21 to −0.39] Resistance+[0.30 to −0.73] Reactance+[0.39 to −0.12] Impedance.

10. The method of claim 9 wherein the percentage of plasma is determined by the formulation:
4.73−0.85 Sex Factor−0.05 Age−0.09 Resistance−0.21 Reactance+0.13 Impedance.

11. The method of claim 1 wherein the percentage of intracellular fluid volume is determined by the formulation:
[448.71 to −171.89]−[5.48 to 29.16] Sex Factor+[3.60 to −0.38]Age+[1.13 to −29.18] Reactance−[0.07 to 9.99] Heart Girth+[8.09 to 1.28] Impedance+[20.87 to −4.27] Phase Angle+[2.83 to 0.10] Electric Volume.

12. The method of claim 11 wherein the percentage of intracellular fluid volume is determined by the formulation:
138.41−17.32 Sex Factor+1.61 Age−14.02 Reactance−5.03 Heart Girth+4.69 Impedance+8.30 Phase Angle+1.46 Electric Volume.

13. The method of claim 1 wherein the percentage of intracellular fluid volume is determined by the total body water less the extracellular fluid volume.

14. An apparatus for determining and monitoring the state of hydration of a horse, camel or other hairy animal by providing power to a transmitting circuit to transmit a signal to electrodes resulting in a potential difference across the electrodes causing a flow of current between the electrodes reflecting the impedance encountered and sending a signal reflecting such information to receiving circuits which pass that data to processing circuits which process that data with other external inputs to produce an output display reflecting an indication of the state of hydration of the animal, comprising:
means for providing a voltage difference across two or more electrodes; means for maintaining said electrodes in a fixed spatial relation and in good electrical contact with the skin of the animal without penetrating the skin of the animal on which said measurements are to be made; means for measuring the impedance of said animal in the region of or between the electrodes; means for transmitting the measured data to processing circuits which process that data with other external inputs to produce an output display reflecting an indication of the state of hydration of the animal, wherein the other external inputs include one or more of the factors for sex, age, electrode gap, and heart girth; and the output display provides indications of one or more of the outputs produced by formulations of the data reflecting the percentage of total body water, the percentage of extracellular fluid volume, the percentage of plasma and the percentage of intracellular fluid volume and the analyzing monitoring unit may be selected from the group of embodiments consisting of software in a computer, hard wired circuits, printed circuits, integrated circuits, microcircuits, microchips, silicon chips, digital chips, analog chips, hybrid chips and combinations of any or all of the members of this group.

15. The apparatus of claim 14 wherein the circuits employed are at least in part hard wired.

16. The apparatus of claim 14 wherein the circuits employed are at least in part printed or integrated circuits.

17. The apparatus of claim 14 wherein the circuits employed are at least in part contained on a chip.

18. The apparatus of claim 14 wherein the percentage of total body water is determined by the formulation:
[66.15 to −50.87]−[8.96 to 16.64] Sex Factor−[0.13 to 0.71] Age+[0.28 to −4.24] Reactance+[1.65 to 0.52] Impedance+[1.16 to 0.26] Electric Volume.

19. The apparatus of claim 14 wherein the percentage of extracellular fluid is determined by the formulation:
[212.02 to −30.21]+[1.68 to −13.04] Sex Factor−[0.08 to 1.44] Age+[1.46 to −0.39] Resistance−[0.76 to 7.23] Reactance+[0.67 to −1.14] Electric Volume.

20. The apparatus of claim 14 wherein the percentage of plasma is determined by the formulation:
[7.97 to 1.48]−[0.12 to 1.57] Sex Factor+[0.03 to −0.13] Age+[0.21 to −0.39] Resistance+[0.30 to −0.73] Reactance+[0.39 to −0.12] Impedance.

21. The apparatus of claim 14 wherein the percentage of intracellular fluid volume is determined by the formulation:
[448.71 to −171.89]−[5.48 to 29.16]Sex Factor+[3.60 to −0.38] Age+[1.13 to −29.18] Reactance−[0.07 to 9.99] Heart Girth+[8.09 to 1.28] Impedance+[20.87 to −4.27] Phase Angle+[2.83 to 0.10] Electric Volume.
